(19) United States
(12) Patent Application Publication
(10) Pub. No.: US 2009/0246234 A1
(43) Pub. Date: Oct. 1, 2009

(54) Therapeutic Treatment Using Niacin for Skin Disorders

(76) Inventor: Benjamin Johnson, Evergreen, CO (US)

Correspondence Address: GLENN L. WEBB;GLENN L. WEBB P.C., P.O BOX 3788, DURANGO, CO, 81302, US

(21) Appl. No.: 12/060,827

(22) Filed: Apr. 1, 2008

(51) Int. Cl. A61K 8/14; A61K 9/127; A61Q 19/08; A61P 17/02

(52) U.S. Cl. 424/401; 424/450

(57) ABSTRACT

Niacin and/or niacinamides are incorporated with liposomes in therapeutic compositions for topical application to prevent or alleviate the conditions and symptoms of skin aging and other histological disorders. In particular, niacin and/or niacinamides are combined with liposomes to increase the penetration of the niacin and/or niacinamides into the deep epidermis and dermis skin layers and to provide hydration of the skin during treatment.

### FIELD OF THE INVENTION

This invention relates to the field of treatments for dermatologic and cosmetic skin disorders.

### BACKGROUND OF THE INVENTION

There are an extensive number of skin care products used for the treatment of skin disorders that may have resulted from aging, environment damage, disease or other factors. In particular, visible signs of skin aging are of concern to many people. These visible signs of skin aging include wrinkling, signs of skin roughness from changes in pore sizes and other indications.

Skin wrinkling is a naturally occurring process that can be aggravated by environmental factors, such as excess sun exposure in particular as well as other environmental factors.

The skin is formed in many layers that are generally considered to form three main layers, epidermis, dermis and subcutaneous layers. The epidermis layer contains skin cells, pigment and proteins. The epidermis layer includes blood vessels, nerves hair follicles and oil glands and provides nutrients to the epidermis. The subcutaneous layer contains sweat glands, hair follicles, blood vessels and fat. Connective tissue with collagen fibers are included in each layer to provide support. Elastin fibers in each layer also provide flexibility and strength.

Aging causes a decrease in the number of pigment-containing cells (melanocytes) while the size of those cells increases. This causes changes in the appearance of the skin including hyperpigmentation and large pigmented spots (age spots, liver spots, lentigos).

Another occurrence in the skin due to aging is the changes in the connective tissue in the skin referred to as elastosis. This is often seen as weather-beaten, leathery appearing skin in individuals who are often outdoors.

Aging also causes the blood vessels in the dermis to become more fragile which can lead to the vessels to flatten and even to bleeding. The vessels (capillaries) in the dermis layer provide the vital nutrition for the epidermis and dermis layers. As the capillaries shrink and weaken, the overall nutrition to those skin layers are affected. The degeneration of the vascular system in this layer is a major factor in skin aging.

The connective tissue, particularly at the junction of the skin layers can also flatten or thin partly due to a decrease in the collagen and from changes in the elastin. This can create wrinkles and roughness in the skin. Also, flattening of the retial cones in this are increases the occurrence of wrinkles and roughness in the skin. The loss of collagen and elastin affect the strength and flexibility of the skin leading to wrinkles, and other histological damage.

Other forms of skin damage are of concern to many people as well. These include damage from acne, rosacea, ages spots, canker sores, hyper pigmentation and disfiguring problems.

There have been a number of attempts to treat skin aging and other histological damage through topical compositions. One product that shows promise are vitamin B₃ compounds. In particular, niacin and niacinamide have been used in topical skin compositions to treat certain skin disorders. However, these compounds cause skin irritation and do not penetrate the epidermis and dermis layers easily. Also, flushing of the skin often occurs from these products. Polar solvents have been used to decrease the irritation to the skin from these products, but decreased the efficacy of these products. Other emollients have been used as well but have failed to increase the penetration of the products into the skin layers or to reduce the irritation and flushing of the skin from these products.

Thus a problem exists in providing topical skin care products containing xanthones and other therapeutic compounds that have increased efficacy in the treatment of many dermatologic and cosmetic skin disorders.

### SUMMARY OF THE INVENTION

The present invention solves these and other problems by providing niacin and niacinamide with liposomes. The present invention may in various embodiments be used to increase the efficacy of the use of niacin and niacinamide for therapeutically and cosmetically treating many skin disorders.

A preferred embodiment of the present invention increases the efficacy of a topical skin care product by increasing the penetration of niacin and/or niacinamide into the skin. Compositions containing niacin and/or niacinamide are coated or mixed with liposomal materials. The effect of niacin and/or niacinamide on the skin occurs not only at the upper levels of the skin but also at the dermal level, so without adequate penetration, they have limited function in the skin. The liposomal niacin and/or niacinamide compound has been shown to greatly increase the penetration of the niacin and/or niacinamide which increases the efficacy of the product.

In a preferred embodiment of the present invention, the use of liposomal niacin and/or niacinamide in a topical application is effective in the treatment of skin wrinkles and other signs of aging. The liposomal niacin and/or niacinamide penetrates deeper into the deep epidermis and dermis layers to stimulate collagen and elastin. This increased stimulation reduces the flaccidity in the connective tissues and increases the strength, flexibility and elasticity of the skin layers. Also, roughness is reduced while the epidermal barrier layer is increased.

Another preferred embodiment of the present invention is useful in treating other signs of skin aging including hyperpigmentation and age spots. The liposomal niacin and/or niacinamide penetrates deeper into the deep epidermis and dermis layers to act as vasodilators to improve the vascularity of the capillaries in the dermis layer. The improved vascularity of the capillaries strengthens the capillaries to reduce hyperpigmentation and age spots that were a result of the weakened capillaries.

Another preferred embodiment of the present invention reduces the irritation to the skin. Compositions containing niacin and/or niacinamide are coated or mixed with liposomal materials to reduce the irritation to the skin. Also the additional hydration of the skin using the liposomal formulation reduces the irritation as well.

These and other features of the present invention will be evident from the ensuing detailed description of preferred embodiments and from the claims.

### DETAILED DESCRIPTION OF PREFERRED EMBODIMENTS

The present invention provides products and methods for increasing the efficacy of treating skin disorders. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding the method of installation. It is to be expressly understood that other products and methods are contemplated for use with the present invention as well.

In accordance with the present invention, vitamin B₃ compounds are incorporated with liposomes in therapeutic compositions for topical application to prevent or alleviate the conditions and symptoms of cosmetic and dermatologic disorders are described as follows. In particular, niacin and niacinamide are combined with liposomes to increase the penetration of those molecules into the skin, to provide hydration of the skin during treatment, and other embodiments and uses.

### Niacin and Niacinamide

Niacin (pyridine-3-carboxylic acid) and the physiologically active form of niacin, niacinamide (pyridine-3-carboxylic acid amide) are necessary to all living cells. They are the essential component of two coenzymes, niacinamide adenine dinucleotide (NAD) and niacinamide adenine dinucleotide phosphate (NADP) which are necessary for cell metabolism. Both of these compounds, when applied directly to the skin impart a roughness to the skin, which decreases the efficacy of these compounds.

The niacin and niacinamide (as well as other derivatives including nicotinic acid and other esters) are believed to increase the vascularity of the capillaries, particularly in the dermis layer but only if they penetrate the epidermis and dermis layers sufficiently. The increase in vascularity of the capillaries increases the flow of blood which increases the flow of nutrients as well as reducing the problems associated with the weakening of the capillaries. Since the weakening of the capillaries is one of the causes of hyperpigmentation and age spots, improving the vascular health of the capillaries will reduce those signs of skin aging.

Niacin and niacinamide also increase the collagen stimulation and elastin stimulation. This reduces wrinkling and skin roughness due to the loss of collagen and elastin and improvise the elasticity, flexibility and strength of the skin layers.

The health of the connective tissues between the layers is also improved by the increase in the stimulation of collagen and elastin at those areas. However, this requires penetration of the niacin and/or niacinamide to those areas in the deep epidermis and dermis layers.

### Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

Preparation of Exemplary Therapeutic Compositions

Accordingly, a preferred embodiment of the present invention provides cosmetic as well as medicinal compositions containing niacin, niacinamide and other vitamin B₃ compounds in liposomal material which when topically administered will substantially improve and alleviate the symptoms of various cosmetic conditions or dermatologic disorders including skin wrinkling, skin lines, age spots, hyperpigmentation and other signs of skin aging and histological skin damage.

The niacin and/or niacinamide of the instant invention may be formulated for topical application in aqueous or non-aqueous solution, gel, lotion, cream or ointment containing 0.1 to 80 percent and preferably from 0.5 to 30 percent by weight of the total composition. Other additives may be used to stabilize or otherwise provide functionality such as solvents, plasticizers, emulsify, stiffen, or other functions. Liposomal lecithin or a liposome substitute or other lipid preparations are added to the above solution with mixing until a uniform consistency is obtained.

To prepare a typical aqueous solution, niacin and/or niacinamide are dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, niacin and/or niacinamide are dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved. When a combination composition is desired retinyl palmitate and/or hydroquinone, for example is added to the above non-aqueous solution. The preferred concentration of retinyl palmitate ranges from 1 to 5%. The concentration of hydroquinone may range from 1 to 5%, but the preferred concentration is 2% by weight of the total composition.

A typical cream or lotion containing mangosteen derivates is prepared by first dissolving the niacin and/or niacinamide in ethanol, acetone, propylene glycol or other solvent. The solution thus prepared is then admixed with commonly available oil-in-water emulsions. BHT or sodium metabisulfite may be added to such emulsions to stabilize the niacin and/or niacinamide. Liposomes or liposome substitutes are added to this solution with mixing until a uniform consistency is achieved.

A typical gel composition is formulated by first dissolving niacin and/or niacinamide in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

Therapeutic Uses

The present invention may in various embodiments be used to increase the efficacy of the use of niacin and/or niacinamide for therapeutically and cosmetically treating many skin disorders.

A preferred embodiment of the present invention increases the efficacy of a topical skin care product by increasing the penetration of niacin and/or niacinamide into the skin. Compositions containing niacin and/or niacinamide are coated or mixed with liposomal materials as described above. The liposomal niacin and/or niacinamide compound has been shown to increase the penetration of niacin and/or niacinamide increases the efficacy of the niacin and/or niacinamide product.

The niacin and/or niacinamide then improves the health of aged and damaged skin. This is due to the increased penetration of the niacin and/or niacinamide into the deep epidermis and dermal skin layers. These compounds act as vasodilators to improve the vascularity of the capillaries. The improved vascularity reduces the hyperpigmentation and age spots due to the weakening of the capillaries. Also, the overall health of the skin is increased by the improved vascularity. The preferred embodiment of the liposomal niacin and/or niacinamide allow deeper penetration of this beneficial activity that has not been possible in prior topical compositions.

The increased penetration of the niacin and/or niacinamide also stimulates the growth of the elastin and collagen in the deep epidermis and dermal skin layers. This reduces the flaccidity of the connective tissues and increases their strength, elasticity and flexibility. Thus, skin wrinkles, skin lines, roughness and other histological damage of the skin are reduced by this deeper penetration.

The effect of niacin and/or niacinamide on the skin occurs at the deep epidermis and dermal level, so without adequate penetration, they have limited function in the skin. These compounds will often irritate the skin and lead to flushing. The liposomal niacin and/or niacinamide compound has been shown to greatly increase the penetration of the niacin and/or niacinamide which increases the efficacy of the product.

In a preferred embodiment of the present invention, the use of liposomal niacin and/or niacinamide in a topical application also may be effective in the treatment of skin wrinkles, skin lines (stretch marks), age spots as well as hyperpigmentation and other histological damage.. The use of niacin and/or niacinamide with liposomes increase the penetration of the niacin and/or niacinamide to increase the vascularity of the capillaries in the dermis layer as well as to stimulate the growth of collagen and elastin in the deep epidermal and dermal layers.

Another preferred embodiment of the present invention increased the hydration of the skin from using niacin and/or niacinamide. Compositions containing niacin and/or niacinamide are coated or mixed with liposomal materials as described above. Liposomes have a natural affinity for water which assists in increasing the moisture in the skin during topical application of the liposomal niacin and/or niacinamide composition.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

1. A method for increasing the transdermal penetration of vitamin B₃ compounds from a topical skin care application, said method comprising the steps of:
providing a vitamin B₃ compound in the skin care application;
providing said vitamin B₃ compound within a liposome compound to increase the transdermal penetration of said compound; and
applying said vitamin B₃ compound within a liposome compound to the skin.

2. The method of claim 1 for increasing the transdermal penetration of vitamin B₃ compounds from a topical skin care application of claim 1 wherein step of providing said compound includes:
providing niacin as said vitamin B₃ compound.

3. The method of claim 1 for increasing the transdermal penetration of vitamin B₃ compounds from a topical skin care application of claim 1 wherein step of providing said compound includes:
providing niacinamide as said vitamin B₃ compound.

4. A method for increasing the vascularity of dermal capillaries, said method comprising the steps of:
providing a topical skin care application;
providing a vitamin B₃ compound within a liposome compound in said topical skin care application to increase the penetration of vitamin B₃ compound into the dermal layer of the skin; and
applying said skin care application topically to the skin.

5. The method of claim 4 for increasing the vascularity of dermal capillaries wherein step of providing said compound includes:
providing niacin as said vitamin B₃ compound.

6. The method of claim 4 for increasing the vascularity of dermal capillaries wherein step of providing said compound includes:
providing niacinamide as said vitamin B₃ compound.

7. A method for increasing the stimulation of collagen and elastin in the dermal skin layers, said method comprising the steps of:
providing a topical skin care application;
providing a vitamin B₃ compound within a liposome compound in said topical skin care application to increase the penetration of vitamin B₃ compound into the dermal layer of the skin; and
applying said skin care application topically to the skin.

8. The method of claim 7 for increasing the stimulation of collagen and elastin in the dermal skin layers wherein step of providing said compound includes:
providing niacin as said vitamin B₃ compound.

9. The method of claim 4 increasing the stimulation of collagen and elastin in the dermal skin layers wherein step of providing said compound includes:
providing niacinamide as said vitamin B₃ compound.

10. A method for treating hyperpigmentation in the skin, said method comprising the steps of:
providing a topical skin care application;
providing a vitamin B₃ compound within a liposome compound in said topical skin care application to increase the penetration of vitamin B₃ compound into the dermal layer of the skin; and
applying said skin care application topically to the skin so that the vascularity of the capillaries in the dermal layer is improved.

11. The method of claim 10 for treating hyperpigmentation in the skin wherein step of providing said compound includes:
providing niacin as said vitamin B₃ compound.

12. The method of claim 10 treating hyperpigmentation in the skin wherein step of providing said compound includes:
providing niacinamide as said vitamin B₃ compound.
